(19) United States
(12) Patent Application Publication
(10) Pub. No.: US 2022/0339207 A1
(43) Pub. Date: Oct. 27, 2022

(54) FAECALIBACTERIUM PRAUSNITZII AND CHRISTENSENELLA BACTERIAL STRAINS FOR THE TREATMENT AND PREVENTION OF BOWEL INFLAMMATION

(71) Applicants: INSTITUT NATIONAL DE RECHERCHE POUR L'AGRICULTURE, L'ALIMENTATION ET L'ENVIRONNENT, Paris (FR); ALMA MATER STUDIORUM - UNIVERSITÀ DI BOLOGNA, Bologna (IT)

(72) Inventors: Philippe LANGELLA, Velizy-Villacoublay (FR); Patrizia BRIGIDI, Bologna (IT); Rebeca MARTIN ROSIQUE, Jouy en Josas (FR)

(21) Appl. No.: 17/629,149

(22) PCT Filed: Jul. 23, 2020
(86) PCT No.: PCT/EP2020/070830

§ 371 Date: Jan. 21, 2022

(30) Foreign Application Priority Data
Jul. 24, 2019 (FR) FR1908385

(51) Int. Cl. A61K 35/74; A23L 33/135

(52) U.S. Cl. CPC A61K 35/74; A23L 33/135; A61K 2035/115

(57) ABSTRACT

The present invention relates to a composition for use thereof in the treatment and/or prevention of an inflammatory bowel disease in an individual, the composition comprising, in a physiologically acceptable medium, at least (I) (a) a first bacterial strain of the Faecalibacterium prausnitzii species, and/or (b) a culture supernatant of a first bacterial strain of the Faecalibacterium prausnitzii species; and (II) (a) a second bacterial strain chosen from the group consisting of Christensenella minuta and Christensenella timonensis, and mixtures thereof, and/or (b) a culture supernatant of a second bacterial strain chosen from the group consisting of Christensenella minuta and Christensenella timonensis, and mixtures thereof.

### FIELD OF THE INVENTION

The present invention relates to a composition for its use in the treatment and/or prevention of inflammatory gastrointestinal diseases, in particular inflammatory intestinal disorders, in an individual.

In particular, it relates to a composition comprising particular bacterial strains and/or their supernatants for its use in the treatment and/or prevention of inflammatory gastrointestinal diseases, in particular inflammatory intestinal disorders, in an individual.

### PRIOR ART

Inflammation is a natural biological process that constitutes a normal part of the response to lesions or infections and contributes to protecting the organism against internal or external attacks.

However, a dysfunction in the mechanisms of inflammation, in particular an inflammation which is persistent or too widespread, could cause painful diseases and put a patient's life in danger. Examples of diseases of this type include skin problems, intestinal disorders, neurological disorders, arthritis and auto-immune diseases. Several of these inflammatory diseases still have no treatment or no suitable treatment.

As a consequence, studying and seeking out novel anti-inflammatory treatment strategies constitutes a major subject in medicine and in biomedical research.

An inflammatory intestinal disorder is a group of disorders characterized by a chronic and recurrent inflammation of the gastrointestinal tract. The most common form in this group is Crohn's disease. Pathogenesis involves inappropriate and continuous activation of the mucosal immune system caused by the presence of intestinal microbiota in a genetically predisposed patient.

There is a constant need for novel substances, in particular probiotics, or for compositions for the treatment and/or prevention of inflammatory gastrointestinal diseases, in particular inflammatory intestinal disorders, in an individual.

Thus, the ability to modulate the immune response of Christensenella, and in particular the bacterial strains Christensenella minuta and Christensenella timonensis, was tested in vitro. In addition, the inventors have identified synergistic anti-inflammatory properties obtained by using a combination of a strain of Christensenella with a strain of Faecalibacterium prausnitzii, a well-known new generation probiotic (NGP).

### DISCLOSURE OF THE INVENTION

The aim of the present invention is to provide a composition for its use in the treatment and/or prevention of an inflammatory gastrointestinal disease in an individual, the composition comprising, in a physiologically acceptable medium, at least a first bacterial strain of the species Faecalibacterium prausnitzii, and a second bacterial strain selected from the group constituted by Christensenella minuta, Christensenella timonensis and/or their supernatants.

A pharmaceutical composition comprising these two strains and/or their supernatants in a physiologically acceptable medium is also envisaged.

### SUMMARY OF THE INVENTION

The aim of the present invention is to describe novel substances, in particular probiotics and/or culture supernatants of probiotics, and compositions for the treatment and/or prevention of inflammatory gastrointestinal diseases in an individual, in particular inflammatory intestinal disorders in an individual.

In the context of the present invention, the terms “prevent” and “prevention” designate the reduction to a lesser degree of the risk or the probability of the occurrence of a given phenomenon, i.e. in the present invention, a gastrointestinal inflammation, in particular an intestinal inflammation.

In the context of the present application, the terms “treat” and “treatment” associated with an inflammatory gastrointestinal disease designate a reduction, or even an interruption, in said inflammatory gastrointestinal disease.

The present invention is based on the discovery by the inventors of the capacity of certain Christensenella bacteria to improve the immunomodulatory properties of a strain of the species Faecalibacterium prausnitzii.

As demonstrated by the inventor's results, the simultaneous use of a first bacterial strain Faecalibacterium prausnitzii, or a supernatant thereof, and of a second bacterial strain selected from the group constituted by Christensenella minuta, Christensenella timonensis and their mixtures, or a supernatant thereof, means that anti-inflammatory results can be obtained that are superior to the simple sum of the combined effects of these strains or their supernatant(s) taken separately.

This is shown in particular by the demonstration of the ability of this combination of strains and/or supernatants to significantly reduce the production of pro-inflammatory molecules, in particular interleukin 8 (IL-8).

Thus, in a first aspect, the present invention concerns a composition for its use in the treatment and/or prevention of an inflammatory gastrointestinal disease in an individual, the composition comprising, in a physiologically acceptable medium, at least:

(I) (a) a first bacterial strain of the species Faecalibacterium prausnitzii, and/or
(b) a culture supernatant of a first bacterial strain of the species Faecalibacterium prausnitzii; and
(II) (a) a second bacterial strain selected from the group constituted by Christensenella minuta, Christensenella timonensis and their mixtures, and/or
(b) a culture supernatant of a second bacterial strain selected from the group constituted by Christensenella minuta, Christensenella timonensis and their mixtures.

In accordance with a particular embodiment, the first bacterial strain is present in the composition in a live, inactive and/or dead form, preferably in a live form.

In accordance with a particular embodiment, the second bacterial strain is present in the composition in a live, inactive and/or dead form, preferably in a live form.

In accordance with a particular embodiment, the composition comprises at least:

(a) a culture supernatant of a first bacterial strain of the species Faecalibacterium prausnitzii; and
(b) a culture supernatant of a second bacterial strain selected from the group constituted by Christensenella minuta, Christensenella timonensis and their mixtures.

In particular, the first bacterium of the species Faecalibacterium prausnitzii is the strain A2-165, deposited in the German Collection of Microorganisms and Cell Cultures in 1996 with number DSM-17677.

In particular, the second bacterial strain is selected from the group constituted by Christensenella minuta DSMZ 22607, Christensenella timonensis DSMZ 102800 and their mixtures.

An individual in accordance with the present invention may in particular be a mammal, more particularly a human being.

In accordance with a particular embodiment, the composition is used in an individual in whom the genus Christensenella represents at least 0.01% of the total number of bacterial genera in their intestinal microbiota.

An inflammatory gastrointestinal disease may in particular be an inflammatory intestinal disorder, more particularly an inflammatory intestinal colic disorder.

Said inflammatory intestinal colic disorder may, in particular, be selected from the group constituted by Crohn's disease, hemorrhagic rectocolitis and pouchitis, in particular from the group constituted by Crohn's disease and hemorrhagic rectocolitis.

In accordance with another embodiment, the composition in accordance with the invention is suitable for oral administration. The composition in accordance with the invention may therefore be in the form of powder or granules, a food product, a drink, a pharmaceutical product, a nutraceutical, a food additive, a food supplement, a dairy product, a capsule or a hard capsule. More particularly, the composition in accordance with the invention may be included in a food supplement.

In accordance with a second aspect, the present invention concerns a pharmaceutical composition comprising, in a physiologically acceptable medium, at least:

(I) (a) a first bacterial strain of the species Faecalibacterium prausnitzii, and/or
(b) a culture supernatant of a first bacterial strain of the species Faecalibacterium prausnitzii; and
(II) (a) a second bacterial strain selected from the group constituted by Christensenella minuta, Christensenella timonensis and their mixtures, and/or
(b) a culture supernatant of a second bacterial strain selected from the group constituted by Christensenella minuta, Christensenella timonensis and their mixtures.

### BRIEF DESCRIPTION OF THE DRAWINGS

FIG. 1 represents the production of interleukin-8 (IL-8) (in pg/mL) (ordinate) in HT-29 cells stimulated by (from left to right): TNF-α alone (YBHI mucin) or in the presence of the strains C. minuta DSMZ 22607 (C. minuta), C. timonensis DSMZ 102800 (C. timonensis), F. prausnitzii A2-165 (F. prausnitzii), C. minuta DSMZ 22607+F. prausnitzii A2-165 (C. minuta+F. prausnitzii) and C. timonensis DSMZ 102800+F. prausnitzii A2-165 (C. timonensis+F. prausnitzii) (abscissa).

### DETAILED DESCRIPTION

The inventors carried out their in-depth studies in order to identify the capacity of a composition comprising a first bacterial strain of the species Faecalibacterium prausnitzii and/or a culture supernatant of this first strain, and a second bacterial strain selected from the group constituted by Christensenella minuta, Christensenella timonensis and their mixtures, and/or a culture supernatant of this second strain, to treat and/or prevent inflammatory gastrointestinal diseases, in particular inflammatory intestinal disorders, in an individual.

In fact, the inventors have unexpectedly determined that a composition in accordance with the invention has the capacity to reduce a gastrointestinal inflammation, in particular an intestinal inflammation, in an individual.

In fact, the present description describes that the composition in accordance with the invention may in particular reduce the concentration of pro-inflammatory molecules such as interleukin 8 (IL-8).

In particular, it has been shown that the combination of a bacterial strain of Christensenella, in particular one or more of the strain(s) Christensenella timonensis and Christensenella minuta, or their supernatant, with a bacterial strain of the species Faecalibacterium prausnitzii, in particular the strain with reference A2-165, or with its supernatant, has anti-inflammatory properties that are superior to those expected from a simple addition of the effects of these strains and/or supernatant employed separately.

It is currently recognized that the commensal probiotic bacterium Faecalibacterium prausnitzii represents approximately 5% of the fecal microbiota in healthy adults (Hold, Schwiertz et al., Appl Environ Microbiol. 2003 July; 69(7):4320-4). The importance of this bacterium is globally recognized, in particular as a contributor to intestinal health in human beings. The relative abundance of F. prausnitzii may also function as an indicator or biomarker of intestinal health in adults.

In this regard, it has been demonstrated that the levels of F. prausnitzii are lower in the feces of individuals suffering from intestinal and metabolic disorders such as inflammatory bowel diseases (IBD), irritable bowel syndrome (IBS), colorectal cancer (CRC), obesity and celiac disease (Balamurugan, Rajendiran et al. J Gastroenterol Hepatol. 2008 August; 23(8 Pt 1):1298-1303, Sokol, Pigneur et al. Proc Natl Acad Sci USA. 2008 Oct. 28; 105(43):16731-6; Neish Gastroenterology. 2009 January; 136(1):65-80; De Palma, Nadal et al. BMC Microbiol. 2010 Feb. 24; 10:63; Furet, Kong et al. Diabetes. 2010 December; 59(12):3049-57; Rajilic-Stojanovic, Biagi et al. Gastroenterology. 2011 November; 141(5):1792-801) as well as in elderly people (van Tongeren, Slaets et al. Appl Environ Microbiol. 2005 October; 71(10):6438-42).

Furthermore, it has also been demonstrated that F. prausnitzii, employed in a live form, exhibits anti-inflammatory effects in vitro and in vivo, which is a sign that this bacterium could contribute to homeostasis in a healthy intestine (Sokol, Pigneur et al. Proc Natl Acad Sci USA. 2008 Oct. 28; 105(43):16731-6).

Christensenella is an anerobic bacterium and its culture necessitates particular equipment and skills. Although it is not considered to be a dominant member of the intestinal microbiota in adults in good health, it represents 8.6% of the bacteria identified in the stools of the mother at the moment of birth in human beings and represents 20% of the bacteria present in samples of newborn meconium (Koenig J E, et al.: Proc Natl Acad Sci USA 2011, 108 Suppl 1:4578-4585). It has also been observed that the members of Christensenellaceae present in fecal samples from healthy volunteers are more numerous than those from patients suffering from an inflammatory gastrointestinal disease in samples from children and youths (Papa E et al.: PLoS One 2012, 7:e39242). These observations have led to the hypothesis that this bacterium is correlated with aging in good health.

Application WO 2016/156527 describes the importance of re-establishing a minimum population of Christensenella in individuals presenting with intestinal inflammatory disease linked to a deficiency of Christensenella in the intestinal microbiota.

Furthermore, applications WO 2018/162738 and WO 2018/162726 teach that the use of particular Christensenella bacteria belonging to species defined as being specifically distinct from those of the genera Christensenella minuta and Christensenella timonensis can combat overweight and obesity in particular, for example, by acting on the presence of visceral fat and on intestinal permeability.

However, the inventors have surprisingly determined that strains of Christensenella, and in particular their supernatants, have the capacity to enhance the anti-inflammatory properties of a strain of Faecalibacterium prausnitzii, and in particular its supernatant. Thus, the inventors have demonstrated the existence of a synergistic effect of a combination of a strain of Christensenella minuta and/or of Christensenella timonensis (or a culture supernatant of such strains) and a strain of Faecalibacterium prausnitzii (or a supernatant of this strain) on the prevention and/or treatment of a gastrointestinal inflammation.

The term “individual” as used in the present text preferably designates a mammal, including a non-human mammal, and more particular a human being. The term “physiologically acceptable medium” designates a medium which is compatible with an organism of the individual to which said composition is to be administered. As an example, this may be a non-toxic solvent such as water. In particular, said medium is compatible with oral administration. A composition in accordance with the invention is preferably suitable for oral administration.

The term “microbiota” should be understood to mean the collection of microorganisms that have colonized an individual and with which that individual cohabits: mostly bacteria, but also viruses, fungi, yeasts and protozoa. The composition of the microbiota differs as a function of the colonized surfaces: there is therefore a distinction between the skin microbiota, the vaginal microbiota, the urinary microbiota, the respiratory microbiota, the ENT microbiota (otorhinolaryngology) and the intestinal microbiota, also known as the intestinal flora, which is by far the largest, with 100 000 billion germs. Thus, the term “intestinal microbiota” means the collection of microorganisms, in particular bacteria, populating the intestine of a given individual.

According to an official definition from the World Health Organization (WHO), the term “Body Mass Index” (BMI) designates an indicator of risks to health associated with excessive weight and with insufficient weight.

TABLE 1
Health risk indicators

| WHO CLASSIFICATION | BMI VALUE (in kg/m²) |
|---|---|
| Underweight | <18.5 |
| Severely underweight | <16.5 |
| Moderately underweight | 16.00-16.99 |
| Slightly underweight | 17.00-18.49 |
| Normal body weight | 18.50-24.99 |
| Overweight | ≥25.00 |
| Pre-obesity | 25.00-29.99 |
| Obesity | ≥30.00 |
| Obesity class I | 30.00-34.99 |
| Obesity class II | 35.00-39.99 |
| Obesity class III | ≥40.00 |

The BMI is calculated by dividing an individual's weight (in kilograms) by the square of their height (in meters).

Composition in Accordance with the Invention

The present invention concerns a composition for its use in the treatment and/or prevention of an inflammatory gastrointestinal disease in an individual, the composition comprising, in a physiologically acceptable medium, at least:

(I) (a) a first bacterial strain of the species Faecalibacterium prausnitzii, and/or
(b) a culture supernatant of a first bacterial strain of the species Faecalibacterium prausnitzii; and
(II) (a) a second bacterial strain selected from the group constituted by Christensenella minuta, Christensenella timonensis and their mixtures, and/or
(b) a culture supernatant of a second bacterial strain selected from the group constituted by Christensenella minuta, Christensenella timonensis and their mixtures.

In particular, the individual defined in accordance with the invention is not overweight, i.e. has a body mass index (BMI) of less than 25.

The term “BMI of less than 25”, designates a BMI of strictly less than 25, more particularly a BMI of less than or equal to 24.99.

In accordance with any one of these embodiments, said individual may have a BMI of less than 25 and greater than or equal to 18.5.

In accordance with a particular embodiment, the composition is used in an individual who is not deficient in Christensenella in the intestinal microbiota.

The term “not deficient in Christensenella” in the intestinal microbiota, as used in the present invention, should be understood to designate an individual for whom the bacteria of the genus Christensenella in their intestinal microbiota represent at least 0.01% of the total number of the bacterial genera detected in their intestinal microbiota, in particular at least 0.1% in number, and especially at least 0.5% of the total number of the bacterial genera detected in their intestinal microbiota. Collected in the stools, the abundance of Christensenella may, for example, be measured by a FISH sequencing test, or by qPCR, or by meta-analysis; these are well known to the person skilled in the art.

In accordance with the present invention, the first bacterial strain of the species Faecalibacterium prausnitzii and/or the second bacterial strain as defined in the invention may be employed independently in a live, semi-active, inactivated or dead form.

In the context of the invention, a bacterial strain in the semi-active form is a microorganism the proliferation capacity of which has been reduced, either temporarily or definitively. The term “semi-active” therefore designates a bacterium with a low physiological activity. This activity may be measured by a longer generation period or exponential growth phase, a slower metabolism or an incomplete physiological response to modifications to the environment, for example. In certain extreme cases, the number of bacteria may be reduced because they can no longer resist changes to the environment.

Thus, in the context of the invention, an “inactivated” bacterial strain is a bacterial strain that is no longer capable of proliferation, either temporarily or definitively.

In the context of the invention, a “dead” bacterial strain is a bacterial strain which is definitively no longer capable of proliferation.

The dead or inactivated bacterial strains may have intact or ruptured cell membranes. Thus, the term “inactivated” also designates extracts or lysates of bacterial strains obtained as detailed below. The dead or inactivated bacterial strains may be obtained by any method known to the person skilled in the art.

An inactivated bacterial strain that is suitable for the invention may be prepared by irradiation, thermal inactivation or lyophilization of a preparation of a bacterial strain. These methods are known to the person skilled in the art.

More particularly, the inactivation of bacterial strains by irradiation may comprise the use of gamma radiation, X rays or exposure to UV. The type of radiation, the intensity, the dose and the exposure time are adjusted by the person skilled in the art as a function of the quantity and nature of the bacterial strains to be inactivated.

Inactivation by lyophilization may be carried out using any method that is known in the field. Advantageously, bacterial strains that have been inactivated by lyophilization may be taken up again into culture.

A bacterial strain in accordance with the invention may be used in a whole form, i.e. essentially in its native form, or in the form of extracts or lysates comprising fractions and/or metabolites of this microorganism. A lysate of this type may in particular be prepared as indicated below.

A lysate in the context of the invention may comprise all or a portion of the fractionated elements and/or metabolites resulting from lysis of the bacterial strain.

A lysate in the context of the invention designates the product obtained following destruction or dissolution of biological cells by a cell lysis phenomenon causing the liberation of the intracellular biological constituents naturally contained in the cells of the bacterial strain under consideration.

In the context of the present invention, the term “lysate” is used indiscriminately to designate the whole of the lysate obtained by lysis of the bacterial strain concerned, or only a fraction thereof.

In the context of the present invention, the term “whole lysate” is used to designate the entirety of the lysate obtained by lysis of the bacterial strain concerned more precisely.

The lysate employed is therefore formed in its entirety or in part by intracellular biological constituents and constituents of the cell walls and membranes.

In accordance with one embodiment, a lysate used for the invention is the entirety of the lysate obtained by lysis of the bacterial strain concerned.

This cell lysis may be carried out with the aid of a variety of technologies, examples being thermal shock, ultrasound, osmotic shock, or mechanical stress such as centrifuging.

In accordance with one embodiment, a culture supernatant of the first bacterial strain as defined above and/or a culture supernatant of the second bacterial strain as defined above may be employed in a composition in accordance with the invention.

The term “culture supernatant” as used in the context of the present invention designates the culture medium in which the bacterial strains have resided during their culture, or extracellular medium.

In accordance with one embodiment, the composition in accordance with the invention comprises at least a culture supernatant of a bacterial strain of the species Faecalibacterium prausnitzii.

In accordance with one embodiment, the composition in accordance with the invention comprises at least one bacterial strain of the species Faecalibacterium prausnitzii.

In accordance with one embodiment, the composition in accordance with the invention comprises at least one bacterial strain of the species Faecalibacterium prausnitzii and at least one culture supernatant of a bacterial strain of the species Faecalibacterium prausnitzii.

In accordance with any one of these embodiments, the bacterial strain of the species Faecalibacterium prausnitzii is a strain of the species F. prausnitzii having anti-inflammatory properties.

In accordance with any one of these embodiments, the bacterial strain of the species Faecalibacterium prausnitzii is the strain with reference A2-165.

In accordance with one embodiment, the composition in accordance with the invention comprises a culture supernatant of at least one bacterial strain selected from the group constituted by Christensenella timonensis, Christensenella minuta and/or their mixtures.

In accordance with one embodiment, the composition in accordance with the invention comprises at least one bacterial strain selected from the group constituted by Christensenella timonensis, Christensenella minuta and/or their mixtures and a culture supernatant of at least one bacterial strain selected from the group constituted by Christensenella timonensis, Christensenella minuta and/or their mixtures.

In accordance with a particular embodiment of the invention, the composition comprises at least:

(a) a culture supernatant of a first bacterial strain of the species Faecalibacterium prausnitzii; and
(b) a culture supernatant of a second bacterial strain selected from the group constituted by Christensenella minuta, Christensenella timonensis and their mixtures.

In accordance with a particular embodiment of the invention, a composition of the invention is suitable for the administration of a daily dose representing 10⁷ to 10¹¹ colony forming units (cfu) of at least one bacterial strain of the species Faecalibacterium prausnitzii, preferably a daily dose equivalent to 10⁹ cfu.

In particular, a composition of the invention is suitable for the administration of a daily dose representing 10⁷ to 10¹¹ colony forming units (cfu) of at least one bacterial strain selected from the group constituted by Christensenella timonensis, Christensenella minuta and/or their mixtures, preferably a daily dose equivalent to 10⁹ cfu.

In the case in which a culture supernatant of any one of the aforementioned bacterial strains is used, a composition in accordance with the invention may in particular comprise, independently, an amount comprised between 0.1% and 99.0% by weight, in particular between 5.0% and 90.0% by weight, in particular 25% to 70% by weight and more particularly 30% to 50% by weight, with respect to the total composition weight.

Thus, a composition in accordance with any one of the aforementioned embodiments may independently comprise an amount comprised between 0.1% and 99.0% by weight, in particular between 5.0% and 90.0% by weight, for example between 10.0% and 90.0% by weight; in particular 25% to 70% by weight and more particularly 30% to 50% by weight of supernatant of a first bacterial strain or a second bacterial strain as defined above, with respect to the total composition weight.

In particular, a composition in accordance with the invention is a pharmaceutical composition.

### Inflammatory Gastrointestinal Diseases

As indicated above, a composition in accordance with the invention such as that described above is used in the treatment and/or prevention of an inflammatory gastrointestinal disease in an individual, more particularly a human being.

In the context of the present invention, an inflammatory gastrointestinal disease is, in particular, an inflammatory intestinal disorder, more particularly an inflammatory intestinal colic disorder. Said inflammatory intestinal colic disorder may, in particular, be selected from the group constituted by Crohn's disease (CD), hemorrhagic rectocolitis (HRC) and pouchitis, in particular Crohn's disease and hemorrhagic rectocolitis.

Crohn's disease and hemorrhagic rectocolitis are two diseases that are characterized by inflammation of the wall of part of the digestive tract, linked to hyperactivity of the digestive immune system.

In particular, inflammatory intestinal disorders, in particular colic disorders, more particularly Crohn's disease (CD), hemorrhagic rectocolitis (HRC) and pouchitis, are diseases which primarily affect young adults, i.e. between 20 and 30 years of age, and which progress by means of flare-ups interspersed with periods of remission. These diseases can affect the entire digestive tract, from the mouth to the anus.

The term “Crohn's disease” designates an inflammatory gastrointestinal disease that can affect the entire digestive tract. Crohn's disease, the cause of which is unknown, is characterized by an inflammation, usually located in the ileum, colon and anus, which is of multi-factorial origin, including a genetic component and the microbiome, inter alia. Crohn's disease typically progresses by means of flare-ups separated by phases which are known as remission phases, which are asymptomatic. The digestive signs are usually a type of diarrhea, abdominal pain or proctological lesions. Diagnosing Crohn's disease requires an esogastric fibroscopy and a coloscopy, with biopsies. Crohn's disease may also be detected using a video capsule, which enables the intestines to be viewed, in particular the small intestine.

The term “hemorrhagic rectocolitis” designates a chronic inflammatory disease of the intestine that affects the distal end of the digestive tract, i.e. the colon and the rectum. It is characterized by continuous lesions that are usually superficial, which start in the rectum and can extend over the entire colon without ever reaching other segments of the digestive tract. It progresses by means of flare-ups interspersed with periods of remission (calm periods with no symptoms).

The term “pouchitis” designates inflammation of the ileal reservoir acquired after a surgeon has carried out an anastomosis between two portions of intestines, i.e. the ileum and the anus. Above all, it concerns patients suffering from hemorrhagic rectocolitis who have undergone a total coloproctectomy.

A composition in accordance with the present invention is in particular provided for the digestive tract, in particular the intestine.

Consequently, a composition in accordance with the invention may be suitable for oral or rectal administration.

In accordance with one embodiment, a composition of the invention is an oral composition, i.e. it is suitable for oral administration to an individual.

A composition of this type may be in the form of a suspension, a tablet, a pill, a capsule, a granule or a powder.

The composition in accordance with the invention for oral administration may be selected from the group constituted by a food product, a drink, a pharmaceutical product, a nutraceutical, a food additive, a food supplement or a dairy product; in particular, it is a food supplement.

In accordance with a particular embodiment, a composition in accordance with the present invention is a food supplement.

A food supplement for oral administration may be present in capsules, hard capsules, soft capsules, tablets, coated tablets, pills, pastes, pastilles, gums, potable solutions or emulsions, a syrup or a gel.

Advantageously, a composition in accordance with the present invention, provided for oral administration, may be provided with a coating which resists gastric juices, in order to ensure that the bacterial strain of the present invention comprised in said composition can pass through the stomach undamaged. Release of the bacterial strain may then occur for the first time in the upper intestinal tract.

A food supplement in accordance with the present invention may furthermore comprise a sweetener, a stabilizer, an antioxidant, an additive, a flavoring agent and/or a colorant. It is formulated using the usual methods for producing coated tablets, hard capsules, gels, controlled release hydrogels, emulsions, tablets or capsules.

A composition in accordance with the present invention may also be in the form of a nutritional composition.

A nutritional composition in accordance with the present invention is in the form of a yogurt, a cereal bar, breakfast cereals, a dessert, a frozen food item, a soup, a pet food item, a liquid suspension, a powder, a tablet, a gum or a piece of candy.

In another embodiment of the present invention, a composition containing the bacterial strain of the invention is administered intrarectally.

In particular, a composition in accordance with the invention that is administered rectally may be in the form of a suppository, an enema or a foam.

A composition in accordance with the invention may furthermore comprise an ingredient selected from: antioxidants, fish oils, DHA, EPA, vitamins, minerals, phytonutrients, a protein, a lipid, probiotics and combinations thereof.

The invention will be described below in more detail with the aid of the following examples which are presented solely by way of illustration.

### Example

### Bacterial Strains, Cell Culture and Growth Conditions

All of the strains described below were obtained from the German DSMZ collection (Leibniz Institute DSMZ). The strains Christensenella minuta DSMZ 22607, Christensenella timonensis DSMZ 102800 and Faecalibacterium prausnitzii A2-165 (deposited in the German Collection of Microorganisms and Cell Cultures under number DSM-17677 in 1996) were cultured at 37° C. in a YBHI medium [brain heart perfusion medium supplemented with 0.5% yeast extract (Difco)] with additions of cellobiose (1 mg/mL; Sigma), maltose (1 mg/mL; Sigma), cysteine (0.5 mg/mL; Sigma) and mucin (1 mg/mL; Sigma) in an anaerobic chamber filled with N₂=85%, CO₂=10% and H₂=5%.

The HT-29 (ATCC HTB-38) (LGC-Standars) cell line was cultured in Dulbecco's Modified Eagle minimum essential medium (DMEM) (Sigma-Aldrich) supplemented with 10% (w/v) of thermo-inactivated fetal calf serum (FCS) (GibcoBRL, Eragny, France) and penicillin G/streptomycin (5000 IU/mL, 5000 μg/mL) (Sigma-Aldrich). The cultures were incubated in 25 cm² tissue culture flasks (Nunc, Roskilde, Denmark) at 37° C. in a 5% CO₂ atmosphere (v/v) to confluence.

### Crossed Feeding Experiments

Single cultures and co-cultures composed of combinations of the listed strains were prepared at time 0. At 24 h, the bacterial samples were centrifuged for 10 minutes at 6000 rpm. The supernatants obtained in this manner were stored at −80° C. in order to proceed with the analysis of their immunomodulating properties in vitro.

### Immunomodulating Properties Using HT-29 Cells

Anti-inflammatory tests were carried out in accordance with the procedure described by Kechaou et al., 2012 (Kechaou N et al.:Appl Environ Microbiol 2012, 79:1491-1499). Specifically, 50 000 HeLa or HT-29 cells per well were seeded into 24-well culture plates (Nunc). Twenty-four hours before bacterial contact, the culture medium was replaced by a medium containing 5% FCS. The experiments were undertaken on the 7th day after seeding, when the cells were at confluence (1.83×10⁶ cells/well). Twenty-four hours before bacterial co-culture (day 6), the culture medium was replaced by a medium containing 5% of heat inactivated FCS and 1% of glutamine. The day of co-culture, 10% of bacterial supernatant was added to DMEM in a total volume of 500 μL. The cells were simultaneously stimulated with human TNF-α (5 ng/mL; Peprotech, NJ) for 6 h at 37° C. in 10% CO₂. After co-incubation, the cellular supernatants were removed and stored at −80° C. until further measurements of the concentrations of interleukin-8 (IL-8) by ELISA (Biolegend, San Diego, Calif.) could be carried out.

These experiments were carried out at least in triplicate.

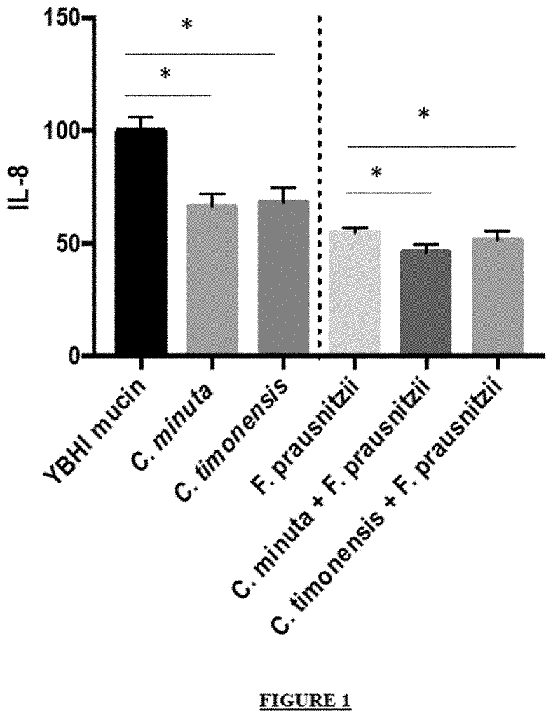

The results obtained are shown in FIG. 1.

These results are expressed as IL-8 (pg/mL) and normalized using the IL-8 produced after co-incubation as the reference value, and PBS as the negative control.

### Statistical Analysis

GraphPad (GraphPad Software, La Jolla, Calif., USA) was used for the statistical analysis. Comparisons were carried out using the non-parametric Kruskal-Wallis test, followed by a Dunn's multiple comparison test. A value for p of less than 0.05 was judged to be significant (*p<0.05; **p<0.01).

### Results

Christensenella minuta DSMZ 22607 and Christensenella timonensis DSMZ 102800 exhibit anti-inflammatory properties in vitro.

The strain with reference F. prausnitzii A2-165 is well known for its immunomodulating properties, and more particularly for its anti-inflammatory effects. In order to determine whether the strains of C. minuta DSMZ 22607 and C. timonensis DSMZ 102800 are capable of modulating the immune response, the inventors carried out in vitro tests on the immunomodulating properties of supernatants of these strains in a model based on the capacity to block the production of IL-8 (a pro-inflammatory cytokine) induced by TNF-α stimulation in HT-29 epithelial cells. Thus, the inventors observed that the two strains are capable of blocking the production of IL-8 in the same manner as the positive control (F. prausnitzii A2-165) (FIG. 1).

C. minuta DSMZ 22607 and C. timonensis DSMZ 102800 enhance the anti-inflammatory properties of the strain F. prausnitzii A2-165 in vitro.

In order to test whether the members of Christensenella were capable of enhancing the anti-inflammatory properties of other members of the intestinal microbiota, co-cultures with F. prausnitzii A2-165 were carried out.

As FIG. 1 also shows, C. minuta DSMZ 22607 and C. timonensis DSMZ 102800 could advantageously, and in a completely unexpected manner, be used to synergistically enhance the anti-inflammatory properties of the strain F. prausnitzii A2-165 in a statistically significant manner.

1. A method for the treatment and/or prevention of an inflammatory gastrointestinal disease in an individual, comprising administration of a composition to said individual, the composition comprising, in a physiologically acceptable medium, at least:
(I) (a) a first bacterial strain of the species Faecalibacterium prausnitzii, and/or
(b) a culture supernatant of a first bacterial strain of the species Faecalibacterium prausnitzii;
and
(II) (a) a second bacterial strain selected from the group consisting of Christensenella minuta, Christensenella timonensis and their mixtures,
and/or
(b) a culture supernatant of a second bacterial strain selected from the group consisting of Christensenella minuta, Christensenella timonensis and their mixtures.

2. The method as claimed in claim 1, characterized in that the first bacterial strain is present in the composition in a live, inactive and/or dead form.

3. The method as claimed in claim 1, characterized in that the second bacterial strain is present in the composition in a live, inactive and/or dead form.

4. The method as claimed in claim 1, comprising at least:
(a) a culture supernatant of a first bacterial strain of the species Faecalibacterium prausnitzii; and
(b) a culture supernatant of a second bacterial strain selected from the group consisting of Christensenella minuta, Christensenella timonensis and their mixtures.

5. The method as claimed in claim 1, characterized in that the first bacterial strain of the species Faecalibacterium prausnitzii is the strain A2-165 deposited in the German Collection of Microorganisms and Cell Cultures in 1996 with number DSM-17677.

6. The method as claimed in claim 1, characterized in that the second strain is selected from the group consisting of Christensenella minuta DSMZ 22607, Christensenella timonensis DSMZ 102800 and their mixtures.

7. The method as claimed in claim 1, characterized in that the individual is a mammal.

8. The method as claimed in claim 1, characterized in that the inflammatory gastrointestinal disease is an inflammatory intestinal disorder.

9. The method as claimed in claim 1, characterized in that the inflammatory intestinal disorder is an inflammatory intestinal colic disorder.

10. The use method as claimed in claim 9, characterized in that the inflammatory intestinal colic disorder is selected from the group consisting of Crohn's disease and hemorrhagic rectocolitis.

11. The method as claimed in claim 1, characterized in that the composition is suitable for oral administration.

12. The method as claimed in claim 1, said composition being in the form of powder or granules, a food product, a drink, a pharmaceutical product, a nutraceutical, a food additive, a food supplement, a dairy product, a capsule or a hard capsule.

13. A pharmaceutical composition comprising, in a physiologically acceptable medium, at least:
(I) (a) a first bacterial strain of the species Faecalibacterium prausnitzii, and/or
(b) a culture supernatant of a first bacterial strain of the species Faecalibacterium prausnitzii;
and
(II) (a) a second bacterial strain selected from the group consisting of Christensenella minuta, Christensenella timonensis and their mixtures,
and/or
(b) a culture supernatant of a second bacterial strain selected from the group consisting of Christensenella minuta, Christensenella timonensis and their mixtures.

14. The pharmaceutical composition as claimed in claim 13, characterized in that the first bacterial strain is present in the composition in a live, inactive and/or dead form.

15. The pharmaceutical composition as claimed in claim 13, characterized in that the second bacterial strain is present in the composition in a live, inactive and/or dead form.

16. The pharmaceutical composition as claimed in claim 13, comprising at least:
(a) a culture supernatant of a first bacterial strain of the species Faecalibacterium prausnitzii; and
(b) a culture supernatant of a second bacterial strain selected from the group constituted by Christensenella minuta, Christensenella timonensis and their mixtures.

17. The pharmaceutical composition as claimed in claim 13, characterized in that the first bacterial strain of the species Faecalibacterium prausnitzii is the strain A2-165 deposited in the German Collection of Microorganisms and Cell Cultures in 1996 with number DSM-17677.

18. The pharmaceutical composition as claimed in claim 13, characterized in that the second strain is selected from the group constituted by Christensenella minuta DSMZ 22607, Christensenella timonensis DSMZ 102800 and their mixtures.

19. The pharmaceutical composition as claimed in claim 13, characterized in that the pharmaceutical composition is suitable for oral administration.

20. The pharmaceutical composition as claimed in claim 13, said pharmaceutical composition being in the form of powder or granules, a food product, a drink, a pharmaceutical product, a nutraceutical, a food additive, a food supplement, a dairy product, a capsule or a hard capsule.
